(19) United States
(12) Patent Application Publication
(10) Pub. No.: US 2013/0066219 A1
(43) Pub. Date: Mar. 14, 2013

(54) Method for Assessing The Efficacy of a Flow-Diverting Medical Device in a Blood Vessel

(71) Applicants: Jingfeng Jiang, Madison, WI (US); Charles Strother, Madison, WI (US)

(72) Inventors: Jingfeng Jiang, Madison, WI (US); Charles Strother, Madison, WI (US)

(21) Appl. No.: 13/229,141

(22) Filed: Sep. 9, 2011

(51) Int. Cl. A61B 5/026

(52) U.S. Cl. 600/504

(57) ABSTRACT

A method for producing a computational flow dynamics model for assessing the efficacy of the deployment of a flow-diverting device in a blood vessel of a patient is provided. Image data of the patient is acquired with a medical imaging system, from which images depicting the blood vessel are reconstructed. A pre-treatment blood vessel model is generated by segmenting the reconstructed images. This pre-treatment blood vessel model is then used to generate a post-treatment, or post-deployment, model of the blood vessel. A post-deployment model of the flow-diverting device is generated and used together with the post-treatment blood vessel model to generate a computational flow dynamics model.

### BACKGROUND OF THE INVENTION

The field of the invention is systems and methods for computational flow dynamics (“CFD”) modeling of medical devices. More particularly, the invention relates to producing CFD models for assessing the effect of flow-diverting devices on cerebral aneurysms.

A cerebral aneurysm is a pathological dilation of a blood vessel in the brain. Cerebral aneurysms result in thin, weak spots on the blood vessel wall, which carry a risk of rupturing the vessel wall.

The conventional endovascular approach for treating cerebral aneurysms is rapidly evolving from coiling, or stent-assisted coiling, to using flow diversion devices, such as densely woven flow-diverting stents. Placing such a low porosity stent across the neck of a cerebral aneurysm diverts flow away from the aneurysm, thereby excluding the aneurysm from the blood stream. Other than intraluminal flow-diverting devices like stents, intrasacular flow-diverting devices could also be deployed within the aneurysm sac to block flow from entering the aneurysm. Consequently, the altered aneurismal hemodynamics could induce thrombosis within the aneurysm sac, stopping its further growth and preventing its rupture. Trials using flow-diverting stents have shown early promising results, while clinical trials of the new intrasacular flow-diverting devices are ongoing

Because these aforementioned flow-diverting devices are used to treat cerebral aneurysms by directly altering the aneurismal hemodynamics, there is increasing interest in the characterization of flow in and around cerebral aneurysms before and after the deployment of a particular device. This characterization is preferably done using a virtual device and using patient-specific computational fluid dynamics (“CFD”) simulations. These CFD simulations have the potential to provide value both as a treatment planning tool and as a tool that can evaluate the efficacy of flow-diverting devices.

In general, CFD simulations operate by removing one or more deployed virtual flow-diverting devices from a bio-fluid domain while the Navier-Stokes equations are solved. Unfortunately, obtaining in vivo image data of a deployed flow-diverting device made of finely woven, small (20-30 micrometer) wires and with small pores (around 100 micrometers in size), with details sufficient for computer modeling, is a very challenging task using current medical imaging techniques. Two practical approaches have been described for virtual deployment of flow-diverting devices into the fluid domain: mechanics-based and parametric/semi-empirical methods.

For mechanics-based methods, once detailed information about an individual patient and a particular device is known, finite element analysis (“FEA”) can be performed to determine detailed geometry of the flow-diverting device after deployment. Examples of the detailed information used in these FEA techniques includes material properties and geometries of both the flow-diverting devices and the vessel wall, connectivity of individual struts and wires of the flow-diverting devices, and relevant boundary conditions. Although it is quite rigorous, numerical difficulties associated with large deformations, such as those greater than twenty percent, during the virtual device deployment, as well as missing subject-specific key information regarding vessel wall characteristics such as material properties and thickness, have been major limiting factors for these mechanics-based approaches. More importantly, because of the computational cost, these methods are not particularly well-suited for applications to clinical problems where compromised, but less computationally-demanding techniques, might potentially be integrated into daily clinical practice.

The parametric techniques are semi-empirical methods. Starting from either a computer-aided design (“CAD”) drawing of an initial flow-diverting device model or a triangulated surface mimicking geometry of the fully expanded device, a pattern is determined by a set of equations and/or other criteria. These governing equations and constraining criteria could be inferred using a number of methods. including performing FEA, image data, and mathematical equations. In particular, through statistical analysis of a reasonable number of cases using ex vivo image data, empirical equations and criteria accounting for the properties of flow-diverting devices including the circumferential or longitudinal spacing and strength of struts, as well as the parent artery information such as bending angles, might be used to derive the final shape of the targeted flow-diverting device. However, to our knowledge, such reliable empirical equations/criteria have not been reported in the peer-reviewed literature.

To date, one of the most sophisticated parametric techniques is based on constrained simplex deformable models using a second-order partial differential equation. The constraints used in this technique can be empirically adjusted to account for a specific stent design. However, all of these methods are based on one of two assumptions. First, it is assumed that flow-diverting devices are expandable, but compliant to the vessel morphology, and second, it is assumed that the flow-diverting device can partially reside outside of the vessel geometry.

Once the geometry of the expanded flow-diverting device is obtained by one of the above-mentioned virtual deployment techniques, computing grids that “subtract” the flow-diverting device from the fluid domain need to be generated. Generally, two types of grids/meshes are commonly used for CFD simulations: body-conforming and embedded. It is worth noting that, for body-conforming grids, an envelope of any particular flow-diverting device needs to be fully contained by the fluid domain, that is, within the vessel walls, and then needs to be subsequently removed from the fluid domain. However, for the embedded techniques, also known as immersed boundary methods, the flow-diverting device is only placed inside a large fluid domain as “solid” references, with special treatment to stop flow around these solid references; thus, the solid need not be removed from the fluid domain.

The weakness and strengthens of the aforementioned approaches for grid/mesh generation are well understood and documented in literature. Generally, both types of CFD grids/meshes containing fine details of a flow-diverting device result in a large number of computing elements/cells, such as more than twenty million cells. These large models are too computationally expensive to make them attractive for routine use in a clinical setting.

It would therefore be desirable to provide a clinically relevant method for virtual deployment of a flow-diverting device. Moreover, it would be desirable to provide such a method that is computationally efficient, thereby allowing its use in clinically relevant times by clinicians to assess the efficacy of flow-diverting device deployments and to plan the deployment of such devices.

### SUMMARY OF THE INVENTION

The present invention overcomes the aforementioned drawbacks by providing a method for virtual deployment of a flow-diverting device that uses a porous media approach to reduce computational cost. This method is capable of automatically generating subject-specific computational flow dynamics (“CFD”) models with the embedment of virtual flow-diverting devices in clinically relevant times, thereby providing a method that supports clinicians in treatment planning and post-treatment evaluation of flow-diverting device deployment in clinically acceptable times.

It is an aspect of the invention to provide a method for producing a computational flow dynamics model for assessing the efficacy of the deployment of a flow-diverting device in a blood vessel of a patient. Image data of the patient is acquired with a medical imaging system, such as a magnetic resonance imaging (“MRI”) system, an x-ray computed tomography (“CT”) system, or an x-ray digital subtraction angiography system, from which images depicting the blood vessel are reconstructed. A pre-treatment blood vessel model is generated by segmenting the reconstructed images. This pre-treatment blood vessel model is then used to generate a post-treatment, or post-deployment, model of the blood vessel. A post-deployment model of the flow-diverting device is generated and used together with the post-treatment blood vessel model to generate a computational flow dynamics model.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flowchart setting forth the steps of an example of a method for producing a computational flow dynamics model for the deployment of a flow-diverting device in a blood vessel of a patient;

FIG. 2 is a flowchart setting forth the steps of an example of a method for generating a post-treatment model of a vessel-of-interest;

FIG. 3 is a flowchart setting forth the steps of an example of a method for decomposing a pre-treatment vessel model into different components, such as an aneurysm component, an affected vessel component, and an unaffected vessel component; and

FIG. 4 is a flowchart setting forth the steps of an example of a method for generating binary masks of portions of a vessel model.

### DETAILED DESCRIPTION OF THE INVENTION

Computational flow dynamics (“CFD”) models containing details of flow diverters are currently too large to be clinically useful. Provided herein is a method for generating a CFD model that uses a porous media approach for the characterization of flow in and around cerebral aneurysms before and after virtual device implantation as an aid for evaluating device efficacy and treatment planning. Generally, an original test device, defined as fully expanded geometry, is deformed to conform to a patient-specific vascular geometry using morphological operations. Mathematical treatments are then applied onto interfaces between the device and the flow surface to mimic flow alterations caused by the device.

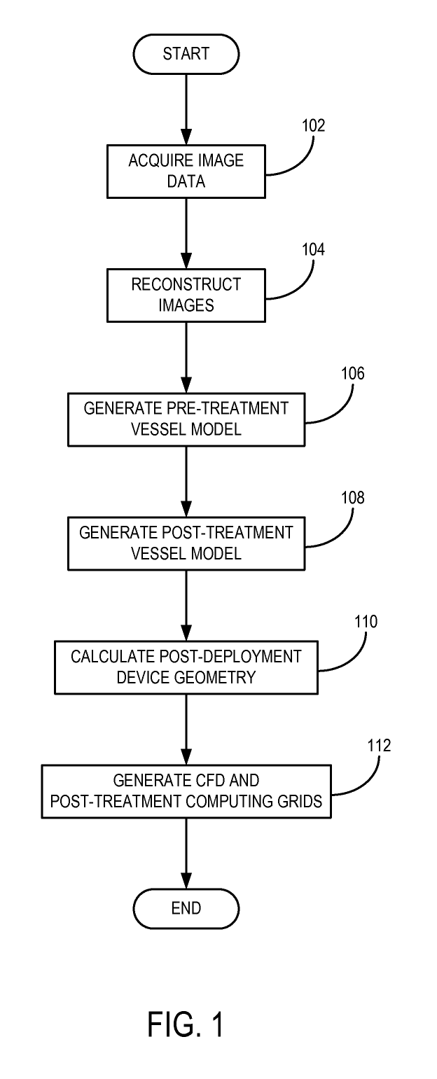

Referring now to FIG. 1, a flowchart setting forth the steps of an example of a method for producing a computational flow dynamics (“CFD”) model for the deployment of a flow-diverting device in a blood vessel of a patient is illustrated. The method beings with the acquisition of image data from the patient using a medical imaging system, as indicated at step 102. The medical imaging system may include a magnetic resonance imaging (“MRI”) system, an x-ray computed tomography (“CT”) imaging system, or the like. From the acquired image data, images of the patient are reconstructed, as indicated at step 104. By way of example, these images may be three-dimensional images that depict the vasculature of interest in the vicinity of a cerebral aneurysm that has been targeted for treatment. Using these reconstructed images, a pre-treatment model of a vessel-of-interest is generated, as indicated at step 106. An example of a vessel-of-interest is the parent vessel of the targeted cerebral aneurysm. By way of example, the pre-treatment model of the vessel-of-interest may be generated using a well-known segmentation technique, such as a marching cubes algorithm. Preferably, this pre-treatment model includes a surface mesh composed of a plurality of surface triangles; however, it is contemplated that the composition of the pre-treatment model will have little influence on the final results so long as the surface mesh is reasonably smooth and dense.

After the deployment of a flow-diverting device, complex biomechanical interactions between the device and the vessel wall result in changes in geometry of the vasculature; thus, it is important to generate a model of the post-treatment, or post-deployment, vessel geometry. Such a post-treatment model of the vessel-of-interest is generated, as indicated at step 108.

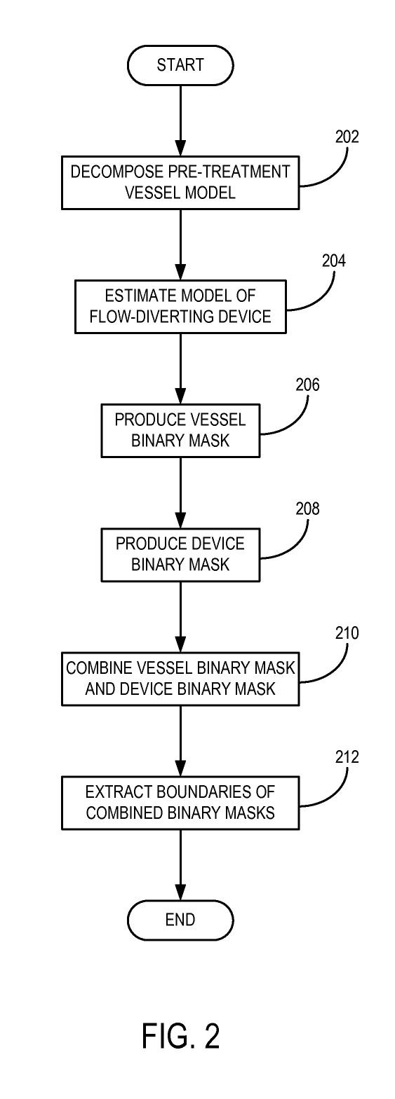

Referring now to FIG. 2, a flowchart setting forth the steps of an example of a method for generating a post-treatment model of a vessel-of-interest is illustrated. The post-treatment vessel model may be generated by first decomposing the pre-treatment model of the vessel-of-interest into different components, as indicated at step 202. By way of example, the vessel model is decomposed into three parts: the targeted aneurysm, the portion of the vessel-of-interest that is affected by deployment of the flow-diverting device, and the portion of the vessel-of-interest that remains unaffected by deployment of the flow-diverting device. By decomposing the vessel-of-interest into these three components, the affected portions of the vessel can be selectively deformed or reconstructed as needed. For example, the geometry of an aneurysm may be altered if an intrasacular device is used, or a portion of the parent vessel may be altered if an intraluminal device is used. Thus, the geometry of the affected portion of the vessel-of-interest can be selectively altered, after which the modified geometry can be reconnected with the other vessel components to create the post-treatment vessel model in a more computationally efficient manner.

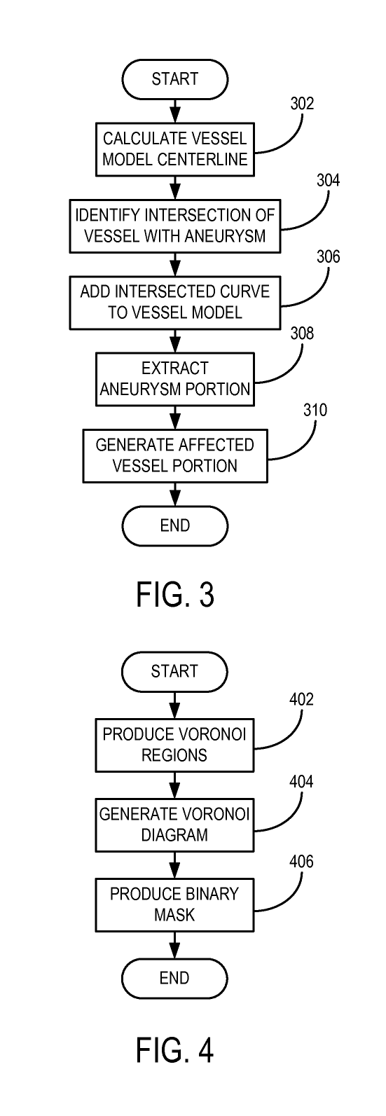

Referring now to FIG. 3, a flowchart setting forth the steps of an example of a method for decomposing a pre-treatment vessel model into different components, such as an aneurysm component, an affected vessel component, and an unaffected vessel component, is illustrated. The pre-treatment vessel model may be decomposed as follows. Normal arteries without aneurysms are assumed to be generally tubular structures that can be constructed about a known centerline. Thus, the centerline of the pre-treatment vessel model is first calculated, as indicated at step 302. The centerline may be calculated as described by L. Antiga and D. A. Steinman, in “Robust and Objective Decomposition and Mapping of Bifurcating Vessels,” IEEE Trans Med Imaging, 2004; 23: 704-713. The calculation of the vessel centerline allows for a determination of the geometry of the tubular blood vessel geometry, and thus allows for a determination of where the vessel intersects the aneurysm portion of the vessel model. Thus, the intersection of the vessel with the aneurysm is identified next, as indicated at step 304. The intersection may be determined using, for example, a collision detection algorithm, such as one that utilized a triangle-triangle intersection test when the pre-treatment vessel model is composed of surface triangles. By identifying the intersection of the vessel and the aneurysm, an intersection curve is determined. This intersection curve may then be added to the vessel wall, as indicated at step 306, to generate an enclosed volume that includes both the affected and unaffected portions of the vessel. In this manner, the aneurysm portion of the model may now be readily extracted, as indicated at step 308.

Once the target aneurysm portion has been extracted the vessel-of-interest can be decomposed into the affected and unaffected portions. In general, the affected portion of the vessel is defined as a portion of the vessel that extends a specified distance beyond the ends of the deployed flow-diverting device. For example, the affected portion can be demarcated by specifying two end points on the vessel-of-interest. In this manner, the affected portion of the vessel is generated, as indicated at step 310, leaving the unaffected portion of the vessel to be those remaining portions of the vessel not including the affected portion.

In the alternative, the pre-treatment blood vessel model may be decomposed using binary masks. An example of a method for producing a binary mask from the pre-treatment model is described below in detail. By way of example, however, two binary masks can be generated: one for the assumed normal tubular blood vessel and the other one for the entire pre-treatment blood vessel, including any aneurysms. By taking the difference between these two binary masks, the aneurysm of interest can be extracted from the blood vessel. The difference between the two masks can then be updated by finding the largest connected volume between the two mask volumes. The affected and unaffected regions of the blood vessel can then be decomposed as provided above.

Referring again to FIG. 2, after the pre-treatment vessel model has been decomposed into the three component parts, a model of the flow-diverting device may be estimated, as indicated at step 204. By way of example, a model of the flow-diverting device geometry may be estimated using a second-order partial differential equation having the form:

$$ρ \frac{{\partial }^{2} P}{\partial {t}^{2}} + γ \frac{\partial {P}_{i} ( t )}{\partial t} - α {f}_{int} ( {P}_{i} ( t ) ) = β {f}_{ext} ( {P}_{i} ( t ) ) ; \qquad ( 1 )$$

where Pᵢ is a point of a simplex mesh, which may be generated from a set of surface triangles; ρ is the mass at the point, Pᵢ; t is pseudo-time; γ is viscous drag; fᵢₙₜ and fₑₓₜ are internal and external forces, respectively; and α and β are associated weighting factors that are used to control the balance between the internal and external forces, respectively. Using the decomposed affected portion of parent vessel as a reference to calculate the internal and external forces, Eqn. (1) can be solved using, for example, a finite difference method to iteratively obtain the geometry of the deployed flow-diverting device. For instance, the initial configuration of the flow-diverting device could be a fully-expanded flow-diverting device, in which the majority of the envelope is outside of the vessel wall. Iteratively, the geometry of the flow-diverting device may be changed until an equilibrium state based on Eqn. (1) is reached. Alternatively, the affected portion of the vasculature may be determined by imaging data or by combining imaging data with finite element analysis.

In general, the affected portion of the vessel geometry should be replaced by a modified geometry that accounts for changes resultant from deployment of a flow-diverting device. Clinical experience has shown that some portions of a flow-diverting device may not be tightly positioned against the vessel wall. As another example, in instances where there is a stenotic segment, some portions of the original vessel wall may be expanded due to the deployment of the targeted flow-diverting devices, where other portions may not. Thus, it is reasonable to assume that the envelope representing the post-treatment vessel geometry will be the union of the pre-treatment model geometry and the deployed virtual flow-diverting device. To facilitate the calculation of this union, a binary mask of the affected vessel portion and a binary mask of the flow-diverting device model are generated, as indicated at steps 206 and 208, respectively. These binary masks may be generated, for example, using a Voronoi diagram technique that will now be described in detail.

Referring now to FIG. 4, a flowchart setting forth the steps of an example of a method for generating binary masks of portions of a vessel model is illustrated. The method begins with the generation of a Voronoi region at each point in the pre-treatment model of the vessel-of-interest, as indicated at step 402. A Voronoi region may be generated as follows. Let ∂Ω represent the volume of the vessel-of-interest and ΩεR³ represent the lumen boundary. In the example provided above, the volume of vessel may be determined from the pre-treatment vessel model as defined by a set of surface triangles containing a set of points, P. Formally, let P={p₁, p₂, . . . , pₙ} be a set of n points of R³. The Voronoi region, V(pᵢ), associated with each point, pᵢ, can be defined as follows:

V(pᵢ)={xε [symbol] ³:∥x−pᵢ∥≦∥x−pⱼ∥,∀j≦n} (2);

where ∥ . . . ∥ is the Euclidean distance.

After the Voronoi regions have been generated, a Voronoi diagram is produced, as indicated at step 404. The Voronoi diagram, Vor (P) is the collection of the Voronoi regions, V(pᵢ), of every point pᵢεP, including their boundary faces. The generated Voronoi diagram may then be used to produce a binary mask of the affected vessel portion, or of the flow-diverting device model estimate, as indicated at step 406. Given a three-dimensional enclosed volume represented by a watertight triangulated surface, such as the pre-treatment vessel model, the Voronoi diagram associated with that surface model can be used to perform a distance test in order to produce a binary mask of the surface model. For example, if a point is within the enclosed volume, the resultant distance is one, while any point outside the volume is zero. A collection of these points in an uniform three-dimensional rectilinear grid create a three-dimensional binary mask that represents the enclosed volume without requiring the use of any analytical functions. Additionally, a binary mask may be generated using, for example, an octree-based bounding volume testing algorithm that operates on a volume, such as the pre-treatment blood vessel model.

Referring again to FIG. 2, after the binary masks of the affected vessel portion and of the flow-diverting device have been produced, they are combined, along with the portion of the blood vessel model corresponding to the unaffected blood vessel, as indicated at step 210. For example, the binary masks may be combined using a Boolean union operation. The post-treatment vessel model may then be produced by extracting the boundaries of combined binary masks, as indicated at step 212. By way of example, the boundaries of the combined masks may be extracted using known image segmentation techniques, such as the marching cubes algorithm.

Referring again now to FIG. 1, the method for assessing a treatment plan for the deployment of a flow-diverting device continues with the calculation of the post-deployment geometry of the flow-diverting device, as indicated at step 110. In general, the methodology for the virtual deployment of a flow-diverting device aims to ensure that the device, when actually deployed, is fully compliant to the post-treatment vessel geometry. Starting from a triangulated surface representing a fully expanded flow-diverting device outside of an artery, this initial surface estimate is deformed to improve the fitting quality to the post-treatment vessel model generated as described above. By way of example, Eqn. (1) may be used to generate the geometry of the flow-diverting device post-deployment. To reduce the computational cost, a simplified method may be used. This simplified method is carried out by a combination of external forces and internal smoothing constraints. The external forces include a deflating force that is computed as the distance vector between the points in the triangulated surface representing the flow-diverting device and it major axis. For instance, the major axis of a cylindrical stent is its centerline, while the major axis of an ellipsoid-like intrasacular device is its long axis. The internal smoothing constraints are based on the assumption that local deformations from the fully expanded state to the final, compliant state are smooth using the classical Laplacian operator. The deformation process is iteratively performed and stopped when all points of the surface representing the flow-diverting device are contained within the vessel lumen.

During the virtual deployment process, it is likely that some areas of the surface representing the deployed device will distorted. If this occurs, distortion indices, such as the large aspect ratio of surface triangles or long edges of surface triangles, can be calculated and used to identify the affected surface triangles. Adaptive refinement of these surface triangles may then be used to obtain a smoothed surface representing the deployed device. By analyzing the local surface distortion, valuable information regarding the changes in local porosity values due to localized changes in pore size can also be obtained.

The computational burden of generating the post-deployment model of the flow-diverting device can further be reduced by only remodeling the segment of a flow-diverting device that fully covers the ostinum of an aneurysm. This partial stent model can be further reduced using an automated algorithm by using a collision detection test, such as the one described above for the automated aneurysm extraction, so that it only covers the neck of the targeted aneurysm.

Once the geometry of the expanded flow-diverting device is obtained by the above-mentioned virtual deployment process, the surface representing the deployed flow-diverting device may be used to generate a computational flow dynamics (“CFD”) grid, as indicated at step 112. For example, a zero-thickness layer where pressure-drops can be added to the governing Navier-Stokes equations to mimic the effect of the actual flow-diverting device may be embedded in the surface. If the thickness of the flow-diverting device needs to be considered, such as when the thickness of the device is not small compared to the size of the vessel, a finite-size layer could be added to the Navier-Stokes equation instead of a zero-thickness layer.

Alternatively, the geometry of the deployed device can be used to facilitate a physical (as opposed to a porous media approach) model of flow-diverting devices. For instance, any deployed stent can first be mapped to an idealized cylinder and then further mapped to a two-dimensional rectangle. For an intrasacular device, the post-deployment device is substantially spherical and can be mapped to an idealized sphere and then mapped onto two unit disks, such as two hemispheres. This process is generally known as harmonic mapping. Then, the actual flow-diverting device can be drawn on the mapped two-dimensional space as a collection of connected lines with appropriate thickness. Finally, all struts or wires representing the actual physical flow-diverting device can be inversely mapped back to the real three-dimensional coordinate system. A mesh generator can remove these struts or wires from the fluid domain during the mesh generation process.

By way of example, a constrained Delaunay triangulation (“CDT”)-based mesh generation algorithm may be used to generate CFD computer grids or meshes for both the porous media approach and the physical approach. Because this approach does not require analytical representations or detailed mechanics of the endovascular devices, it is contemplated that it may serve as a rapid grid generation method for producing computer models in a clinical environment to understand the effects of the flow-diverting devices.

Using the aforementioned method, the effects of a flow-diverting device can be evaluated in clinically relevant times, thereby providing a tool for clinicians to plan the treatment of an aneurysm by assessing the deployment of a particular device, or to evaluate the efficacy of the deployment of a given flow-diverting device.

Any hardware platform suitable for performing the processing described herein is suitable for use with the technology. Non-transitory computer-readable storage media refer to any medium or media that participate in providing instructions to a central processing unit (“CPU”), a processor, a microcontroller, or the like. Such media can take forms including, but not limited to, non-volatile and volatile media such as optical or magnetic disks and dynamic memory, respectively. Examples of non-transitory computer-readable storage media include a floppy disk; a hard disk; magnetic tape; any other magnetic storage medium; a CD-ROM disk; digital video disk (“DVD”); any other optical storage medium; random access memory (“RAM”), including static RAM (“SRAM”) and dynamic RAM (“DRMA”); read only memory (“ROM”), including programmable ROM (“PROM”), erasable PROM (“EPROM”), and an electrically erasable PROM (“EEPROM”); and any other memory chip or cartridge.

The present invention has been described in terms of one or more preferred embodiments, and it should be appreciated that many equivalents, alternatives, variations, and modifications, aside from those expressly stated, are possible and within the scope of the invention.

1. A non-transitory computer readable storage medium having stored thereon a computer program comprising instructions that when executed by a processor causes the processor to:
a) receive a medical image acquired with a medical imaging system and that depicts a blood vessel of a patient;
b) generate a pre-treatment blood vessel model that includes a volume of a normal portion of the blood vessel and a volume of an abnormal portion of the blood vessel by segmenting the received medical image;
c) generate a post-treatment blood vessel model that includes a volume of a normal portion of the blood vessel and a volume of an abnormal portion of the blood vessel as affected by a flow-diverting device using the pre-treatment vessel model generated in step b);
d) calculate a post-deployment flow-diverting device model using the post-treatment blood vessel model generated in step c); and
e) generate a computational flow dynamics model using the post-treatment blood vessel model generated in step c) and the post-deployment flow-diverting device model calculated in step d).

2. The non-transitory computer readable storage medium as recited in claim 1 in which step c) includes decomposing the pre-treatment blood vessel model into components associated with the normal portion of the blood vessel and the abnormal portion of the blood vessel.

3. The non-transitory computer readable storage medium as recited in claim 2 in which the components associated with the abnormal portion of the blood vessel include a component corresponding to an aneurysm, and in which the components associated with the normal portion of the blood vessel includes a portion of the blood vessel affected by the flow-diverting device and a portion of the blood vessel unaffected by the flow-diverting device.

4. The non-transitory computer readable storage medium as recited in claim 2 in which step c) further includes estimating a model of the flow-diverting device.

5. The non-transitory computer readable storage medium as recited in claim 4 in which step c) further includes generating a binary mask from the estimated model of the flow-diverting device and generating a binary mask of the pre-treatment blood vessel model corresponding to a portion of the normal portion of the blood vessel affected by the flow-diverting device.

6. The non-transitory computer readable storage medium as recited in claim 5 in which the binary masks are generated by producing Voronoi regions at locations in the estimated model of the flow-diverting device and the portion of the normal portion of the blood vessel affected by the flow-diverting device.

7. The non-transitory computer readable storage medium as recited in claim 6 in which a Voronoi diagram for the estimated model of the flow-diverting device is formed from the corresponding Voronoi regions, and in which a Voronoi diagram for the portion of the blood vessel affected by the flow-diverting device is formed from the corresponding Voronoi regions.

8. The non-transitory computer readable storage medium as recited in claim 7 in which the binary masks are generated using the formed Voronoi diagrams.

9. The non-transitory computer readable storage medium as recited in claim 5 in which step c) further includes combining the generated binary masks.

10. The non-transitory computer readable storage medium as recited in claim 9 in which step c) further includes generating the post-treatment blood vessel model by extracting a boundary of the combined binary masks.

11. The non-transitory computer readable storage medium as recited in claim 5 in which the binary masks are generated using an octree-based bounding volume testing algorithm.

12. The non-transitory computer readable storage medium as recited in claim in which decomposing the pre-treatment blood vessel model includes calculating a centerline of the blood vessel.

13. The non-transitory computer readable storage medium as recited in claim 12 in which the calculated centerline of the blood vessel is used to estimate a tubular volume of the blood vessel.

14. The non-transitory computer readable storage medium as recited in claim 13 in which decomposing the pre-treatment blood vessel model includes generating a binary mask from the pre-treatment blood vessel model and a binary mask from the estimated tubular volume of the blood vessel, and by performing a subtraction between the binary masks.

15. The non-transitory computer readable storage medium as recited in claim 13 in which decomposing the pre-treatment blood vessel model includes identifying an intersection of the blood vessel and an aneurysm, and thereby calculating an intersection curve.

16. The non-transitory computer readable storage medium as recited in claim 15 in which decomposing the pre-treatment blood vessel model includes extracting an aneurysm component using the calculated intersection curve.

17. The non-transitory computer readable storage medium as recited in claim 15 in which decomposing the pre-treatment blood vessel model includes selecting end points that define a portion of the blood vessel affected by the flow-diverting device.

18. The non-transitory computer readable storage medium as recited in claim 1 in which step e) includes estimating local porosity parameters based on a distortion of the post-deployment flow-diverting device model.

19. The non-transitory computer readable storage medium as recited in claim 1 in which step c) includes decomposing the pre-treatment blood vessel model into the normal portion and the abnormal portion using Voronoi regions produced at locations in the pre-treatment blood vessel model.
